(19) United States
(12) Patent Application Publication
(10) Pub. No.: US 2003/0190374 A1
(43) Pub. Date: Oct. 9, 2003

(54) Methods of using silica hydride mineral

(76) Inventor: Clinton H. Howard, Irving, TX (US)

Correspondence Address: CARSTENS YEE & CAHOON, LLP, P O BOX 802334, DALLAS, TX, 75380

(21) Appl. No.: 10/116,371

(22) Filed: Apr. 4, 2002

(51) Int. Cl. A61K 33/00

(52) U.S. Cl. 424/724

(57) ABSTRACT

The exact health benefits of silica hydride minerals, traditionally found in glacial streams, have long been the subject of speculation. Geochemical analysis indicates that such colloidal silica hydrides in water possess a silica-water interface that provides a hydrated surface and adsorbs other elements or compounds such as potassium, iron, magnesium, lithium, calcium, and hydrogen. Dietary supplements with similar properties have been formulated. When the silica-water interface of such compounds is saturated with reduced hydrogen, the compounds take on an overall negative charge and act as a reducing agent or antioxidant when in solution. When consumed, hydride ions introduced into the body by the silica hydride supplement donate electrons to body fluids. With proper dosages, the benefits of consuming silica hydride include reduction of lactic acid build-up, increasing cellular hydration, reduction of free radical damage, enhancement of mitochondrial bioenergetic capacity, increasing antioxidant activity, and enhancing the properties of drinking water.

### TECHNICAL FIELD OF THE INVENTION

This invention relates to methods of using silica hydride minerals. More particularly, this invention relates to methods of using silica hydride minerals that have beneficial effects on lactic acid buildup during exercise, cellular hydration, free radical damage, mitochondrial bioenergetic capacity, antioxidant activity, and the suitability of water for conversion into optimal cellular body fluids.

### BACKGROUND OF THE INVENTION

Amorphous silicate minerals, many of which are in the nanoparticle size range, were once common in natural water sources and abundant in glacial stream waters. Not only do the silica mineral particles bond water and other elements for transport; they also can be adsorbed with reduced hydrogen, which releases electrons, providing antioxidant or reducing potential to surrounding fluids.

In one region of West Pakistan the people are known to enjoy excellent health and amazing longevity. A team of cardiologists found the heart health of the people to be exceptionally good and evidence of the people's delayed aging. The cardiologists attributed the good health and longevity in significant part to the abundance of colloidal silicate minerals in the glacial streams the people used for irrigation of food crops and drinking water.

Geochemical analysis indicates that colloidal silicate minerals display a variety of properties, including the formation of structured water around the silica-water interface, which provides a hydrated surface that adsorbs elements or compounds such as potassium, iron, magnesium, lithium, calcium, and hydrogen. FIG. 1 illustrates an example of the silica-water interface and the concentric structured water arrangement about the interface with the adsorption of elements within the layers.

From silicate analogs, it is possible to formulate dietary supplements that are similar to the colloidal silicate minerals found in glacial waters and retain the geo-physical properties inherent to these minerals. An example of such synthesized silicate analogs is a silica hydride formula sold under the trademark Microhydrin® (a proprietary formula manufactured by Flanagan Technologies, Inc., Cottonwood, Ariz.). Substances possessing the characteristics and functions described in this application, such as Microhydrin®, have assumed many names. For example, in addition to being called silica hydrides, such substances are known as amorphous silicate minerals, silicate particles, silicates, colloidal silicate minerals, silicate analogs, synthesized silicate analogs, functional silicate nanocolloids, dielectric interstitial hydrides, dietary silicate supplements, or dietary silicate antioxidants. Considering the many labels afforded this class of substances, the characteristics and functions of supplements must necessarily determine whether a particular supplement falls within the class.

Referring again to FIG. 1, the particle's silica-water interface can be saturated with reduced hydrogen, or hydride (H—) ions, and takes on an overall negative charge. In such cases, the particle then acts as a reducing agent or antioxidant when in solution (standard reduction-oxidation potential, −550 mV). It is capable of providing literally trillions of hydride ions able to donate electrons into body fluids. Electrons, which Albert Szent-Gyorgyi called the “fuel of life,” are abundantly available in inorganically grown raw vegetables, fruits, and grains, but are deficient in our modem diet of over-cooked, acidic, or highly oxidized foods, beverages, and drinking water. In silica hydride minerals, the structured water around the silica-water interface stabilizes electron transfer. Such specific silicate interactions could play a substantial role in many biological processes by enhancing salvation properties and ion and water transport and by providing free radical antioxidant protection.

Such electron deficiencies resulting from inadequate diet have a derogatory impact on specific biological processes such as lactic acid build-up, cellular hydration, damage from free radicals, mitochondrial bioenergetic capacity, antioxidant activity, and suitability of drinking water for conversion into optional cellular body fluids. Therefore, a need exists for a method of counter balancing these electron deficiencies and, as a result, enhancing each of these biological phenomena.

### SUMMARY OF THE INVENTION

The present invention identifies certain beneficial health effects of silica hydride minerals and the effective doses necessary to achieve desired results. With proper dosages, the benefits of using silica hydride minerals as a dietary supplement include: reduction of lactic acid buildup during and after exercise, increasing cellular hydration, reduction of free radical damage, enhancement of mitochondrial bioenergetic capacity, increasing antioxidant activity, and enhancing the suitability of water for conversion into optimal cellular body fluids.

### BRIEF DESCRIPTION OF THE DRAWINGS

The novel features believed characteristic of the invention are set forth in the appended claims. The invention itself, however, as well as a preferred mode of use, further objects and advantages thereof, will best be understood by reference to the following detailed description of an illustrative embodiment when read in conjunction with the accompanying drawings, wherein:

FIG. 1 is an enlarged view of a hypothetical silica hydride particle showing the silica-water interface and other adsorbed elements;

FIG. 2 is a chart showing the reduction in lactic acid buildup affected by consumption of silica hydride;

FIG. 3 is a chart of the increase in total body water affected by consumption of silica hydride;

FIG. 4 is a chart of the increase in intracellular water affected by consumption of silica hydride;

FIG. 5 is a chart of the increase in body cell mass affected by consumption of silica hydride;

FIG. 6 is a chart of the increase in extracellular water affected by consumption of silica hydride;

FIG. 7 is a chart of the reduction in free radical damage affected by consumption of silica hydride;

FIG. 8 is a chart of the increase in NADH production affected by silica hydride within in vitro assays;

FIG. 9 is a chart of the increase in NADH production in liver cells by introduction of silica hydride;

FIG. 10 is a chart of the increase in mitochondrial membrane potential in liver cells affected by introduction of silica hydride;

FIG. 11 is a chart of the inhibition of epinephrine oxidation by the super oxide free radical in the standard assay for super oxide dismutase activity;

FIG. 12 is a table reflecting oxidation/reduction potential values of various water samples, some including silica hydride;

FIG. 13 is a table reflecting the oxidation/reduction potential values of various known antioxidants, including silica hydride;

FIG. 14 is a chart reflecting the desired decrease in saliva pH affected by consumption of silica hydride;

FIG. 15 is a chart reflecting the desired decrease in Saliva rH2 affected by consumption of silica hydride;

FIG. 16 is a chart reflecting the desired decrease in Blood Resistivity affected by consumption of silica hydride;

FIG. 17 is a chart reflecting the desired decrease in Urine Resistivity affected by consumption of silica hydride;

FIG. 18 is a chart reflecting the desired increase in Urine pH affected by consumption of silica hydride;

FIG. 19 is a chart reflecting the desired increase in Urine rH2 affected by consumption of silica hydride;

FIG. 20 is a chart reflecting the desired decrease in Saliva Resistivity affected by consumption of silica hydride.

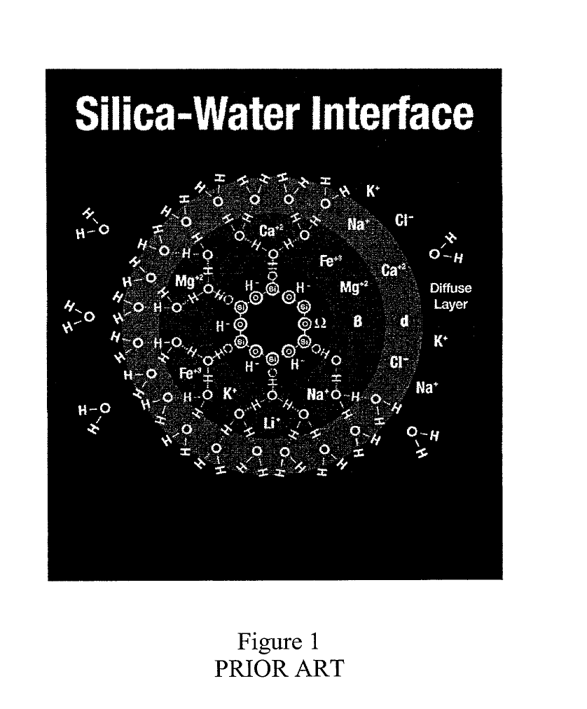

### DETAILED DESCRIPTION OF THE DRAWINGS

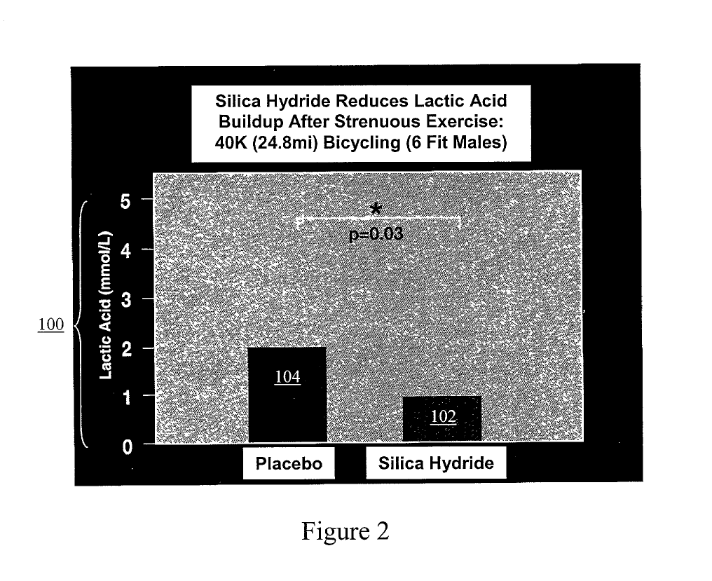

FIG. 2 charts the results of a study of blood lactic acid (lactate) levels 100 in six male cyclists during a 40K (24.8 mi) timed bicycle ride at maximum speed. In this embodiment of an effective dose, subjects received four 250 mg capsules of a silica hydride mineral or placebo each day, taking one in the morning, two at noon and one in the evening during the week prior to testing and during the week of testing. Subjects refrained from all other non-prescribed supplements during the testing period. Additionally, subjects took two 250 mg capsules with water thirty minutes before the start of exercise. Blood lactate was measured before, and five minutes after, each exercise session. During strenuous exercise, the blood lactate levels 100 of the group taking silica hydride 102 were significantly decreased as compared to the placebo group 104, from about 2 to about 1 mmol/L.

Typically, lactic acid accumulates during strenuous or prolonged exercise. It causes pain, limits endurance, and is a common problem for athletes and people who lift weights, play sports, or do physical exercise for an extended time. Decreased lactate levels observed immediately after strenuous exercise indicate the silica hydride mineral's ability to help in providing a direct energy source (ATP production) to cellular function. An ergogenic energy function, such as this, is one that occurs when a substance enhances biochemical energy without introducing additional carbohydrates or calories to the diet.

The dose of silica hydride for reduction of lactic acid build-up described above calls for 250 mg, four times per day. It will be understood by those skilled in the art, however, that a wide range of doses, ascertainable without undo experimentation, produce similar beneficial effects to a greater or lesser degree. Alternative embodiments for reduction of lactic acid build-up include ingesting silica hydride in amounts as small as about 10 mg per day, or amounts as large as about 5000 mg per day. In addition, the doses may be ingested any number of times per day, as infrequently as once, or several more than the above-mentioned four times per day. Individuals may also find it necessary to increase or decrease the amount of silica hydride ingested to achieve desired results. For example, an individual may find 75 mg of silica hydride per day more beneficial than 10 mg. Similarly, one may find that increasing the dosage to 150 mg per day, 250 mg per day, or even 400 mg per day, and so on, achieves progressively better results. On the other hand, where one individual sees peak results taking 5000 mg per day at various intervals, another may see similar results by ingesting 3500 mg per day at a different set of intervals. Likewise, it may be advantageous to limit the dose, say from 3500 mg per day to 2000 mg per day, or to 1000 mg per day, or even to 750 mg per day, in order to see if the same results may be obtained at lower dosage levels.

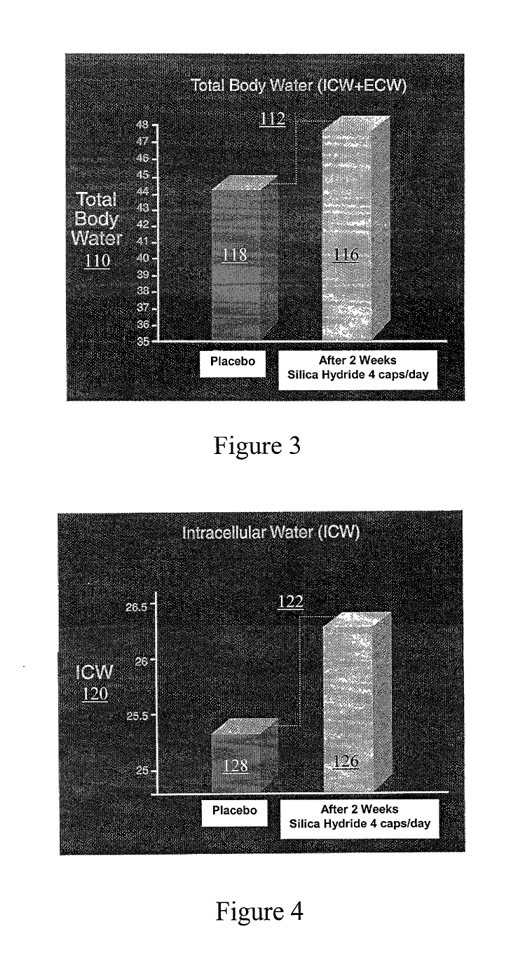

FIGS. 3 through 6 show the results of a study where consumption of silica hydride mineral increased intra- and extra-cellular hydration as compared to a placebo group. Hydration of the body was measured using the RJL Bioelectrical Impedance Analyzer, developed by R. J. Liedtke, which measures body hydration based on nutritional status. In the study, seven subjects received four 250 mg capsules of silica hydride mineral per day for two weeks and were crossed over receiving four capsules per day of a placebo.

Body Cell Mass and Intracellular Water are assessments of intracellular volume and water inside the cell, respectively. Intracellular Water makes up approximately 60% of the total body water of healthy adults. Intracellular Water, as an indicator of cell integrity, is found to be higher in babies but decreases as adults age or lose body cell mass. Healthy tissue cells hold water within the cells and have higher anabolic (building up) function rather than catabolic (breaking down) function.

FIG. 3 charts the value for Total Body Water (TBW) (110), the sum of intracellular (ICW) and extracellular water (ECW), which increased by 2.7% (112) when consuming silica hydride. The changes observed in TBW when subjects consumed the silica hydride mineral (116), compared to the placebo (118), showed statistical significance using a student's test for small sample groups.

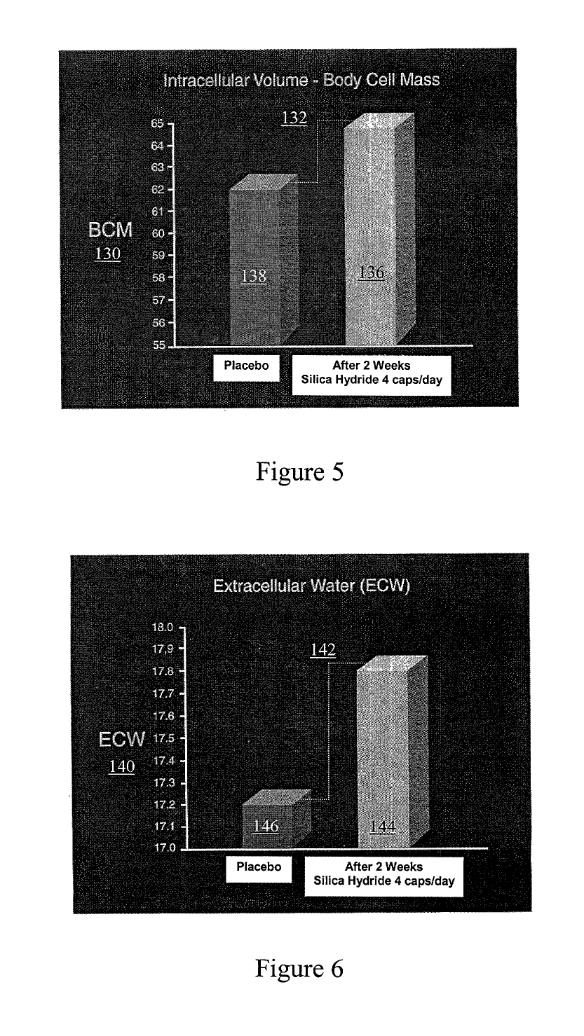

FIG. 4 shows the consumption of silica hydride also increased ICW (120), the most sensitive indicator of nutrition and metabolic state, of the silica hydride consuming group (126) over the placebo group (128), by 2.7% (122). As indicated by FIG. 5, Body Cell Mass (BCM) (130), or intracellular volume, another indicator of water within cells, also showed a 2% increase (132) during supplementation with silica hydride (136) compared to the placebo (138). Similarly, as indicated by FIG. 6, the increase in volume of water observed in ECW values (140), was also statistically significant, about 3.0% (142), when consuming silica hydride (144) as compared to taking the placebo (146).

The dose of silica hydride for increasing cellular hydration described above calls for 250 mg, four times per day. It will be understood by those skilled in the art, however, that a wide range of doses, ascertainable without undo experimentation, produce similar beneficial effects to a greater or lesser degree. Alternative embodiments for increasing cellular hydration include ingesting silica hydride in amounts as small as about 10 mg per day, or amounts as large as about 5000 mg per day. In addition, the doses may be ingested any number of times per day, as infrequently as once, or several more than the above-mentioned four times per day. Individuals may also find it necessary to increase or decrease the amount of silica hydride ingested to achieve desired results. For example, an individual may find 75 mg of silica hydride per day more beneficial than 10 mg. Similarly, one may find that increasing the dosage to 150 mg per day, 250 mg per day, or even 400 mg per day, and so on, achieves progressively better results. On the other hand, where one individual sees peak results taking 5000 mg per day at various intervals, another may see similar results by ingesting 3500 mg per day at a different set of intervals. Likewise, it may be advantageous to limit the dose, say from 3500 mg per day to 2000 mg per day, or to 1000 mg per day, or even to 750 mg per day, in order to see if the same results may be obtained at lower dosage levels.

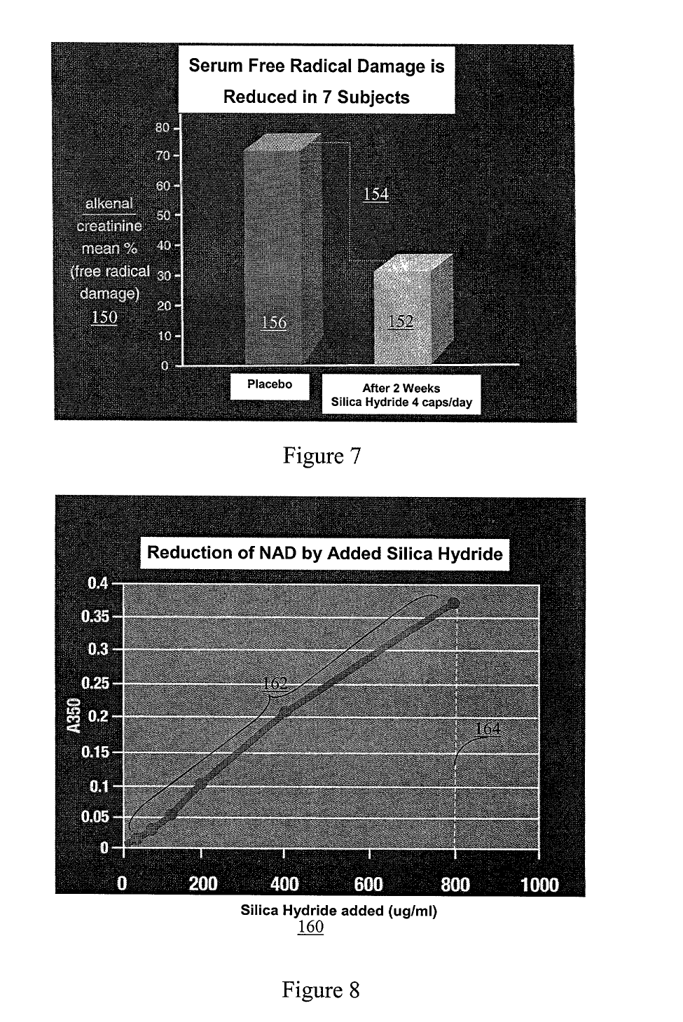

FIG. 7 shows the results of testing to observe the effect of silica hydride mineral consumption on protection against free radical damage. The test of free radical damage was a double blind placebo controlled crossover pilot study conducted on seven subjects who received four 250 mg capsules per day of silica hydride for two weeks and received four capsules per day of a placebo for two weeks. Urine alkenal/creatinine ratios reflect free radical damage (150). The ratios were measured during silica hydride mineral supplementation (152). A 43% decrease (154) in free radical damage was observed as compared to the placebo group (156). The results demonstrate the ability of silica hydride to protect against serum alkenals. Alkenals are the oxidative products of serum lipid peroxides occurring from free radical attacks on cellular lipid membranes and lipoproteins. They are indicators of free radical damage in the body associated with a higher risk of age related diseases.

The dose of silica hydride for reduction of free radical damage described above calls for 250 mg, four times per day. It will be understood by those skilled in the art, however, that a wide range of doses, ascertainable without undo experimentation, produce similar beneficial effects to a greater or lesser degree. Alternative embodiments for reduction of free radical damage include ingesting silica hydride in amounts as small as about 10 mg per day, or amounts as large as about 5000 mg per day. In addition, the doses may be ingested any number of times per day, as infrequently as once, or several more than the above-mentioned four times per day. Individuals may also find it necessary to increase or decrease the amount of silica hydride ingested to achieve desired results. For example, an individual may find 75 mg of silica hydride per day more beneficial than 10 mg. Similarly, one may find that increasing the dosage to 150 mg per day, 250 mg per day, or even 400 mg per day, and so on, achieves progressively better results. On the other hand, where one individual sees peak results taking 5000 mg per day at various intervals, another may see similar results by ingesting 3500 mg per day at a different set of intervals. Likewise, it may be advantageous to limit the dose, say from 3500 mg per day to 2000 mg per day, or to 1000 mg per day, or even to 750 mg per day, in order to see if the same results may be obtained at lower dosage levels.

Another benefit of consumption of silica hydride is enhancement of the bioenergetic capacity of the mitochondria. Such enhancement occurs by promoting formation of NADH, which enhances production of ATP, the principal mitochondrial energy source, as well as by enhancing the membrane potential of the mitochondria.

Hydrogen is one of the most important elements donating an electron, an electron pair, or its proton to reduction/oxidation reactions of numerous enzymes and intermediates within the cell's metabolic pathways. Some of those reactions lead to production of ATP, the principal energy source of the mitochondria. In particular, ATP production depends on the production of NADH. NADH carries reduced hydrogen, generated by the Kreb's Cycle, into the mitochondria for the electron transport chain reactions that will ultimately create a molecule of water and ATP.

FIG. 8 shows the results of tests of in vitro assays designed to measure the direct conversion of NAD⁺ to NADH by the addition of silica hydride mineral. The sequential addition of increasing concentrations of silica hydride (160), up to at least 800 micrograms/ml (164), to NAD⁺ shows a linear increase in NADH production (162) as measured by its absorption at 350 nm.

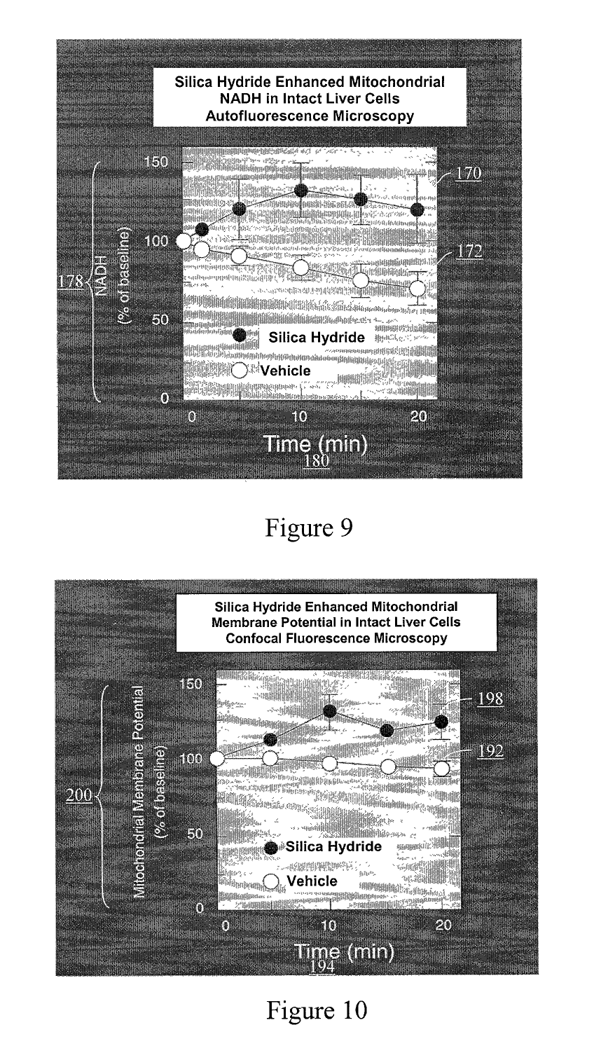

Silica hydride mineral has also proven to enhance NADH production in other tests. FIG. 9 shows measurements of mitochondrial NADH in cultures of intact living hypatocytes. Silicia hydride mineral was introduced to the cultured 90% viable rat hepatocytes. A laser scanning confocal microscope was used to visualize the blue auto-fluorescence of mitochondrial NADH. Under the conditions used, auto-fluorescence arises primarily from mitochondrial NADH. Oxidation of NADH to NAD⁺ causes loss of fluorescence since only NADH is fluorescent, not NAD⁺.

FIG. 9 summarizes data from three silica hydride mineral and three vehicle (control) experiments. In the silica hydride mineral group, NADH (178) increased 20% (170) over 20 minutes (180), while the vehicle group showed that NADH fluorescence (178) decreased by about 30% (172). These experiments demonstrate the promotion of electron transfers to NAD⁺ in intact living hepatocytes. Moreover, the silica hydride mineral prevented the spontaneous oxidation (or bleaching) of NADH that generally occurs during incubation (see vehicle plot (172)), thereby indicating a continuous recharging of the pyridine nucleotide (NADH).

In FIG. 10, mitochondrial membrane potential (200) was monitored using overnight cultured hepatocyes similar to the NADH experiment. The cultured hepatocytes were loaded for 20 minutes (194) with the fluorescent probe, tetramethylrhodamine methylester (TMRM). The medium was adjusted to pH 7.4. In these experiments, an increase of the mitochondrial fluorescence of TMRM represents an increase of mitochondrial depolarization (more negative membrane potential). The line graph summarizes data from three silica hydride mineral, and four vehicle experiments. In the vehicle group, TMRM fluorescence decreased by about 6% (192) over twenty minutes (194). In the silica hydride group, TMRM increased about 25% (198). These experiments demonstrate that silica hydride minerals enhance mitochondrial membrane potential in intact living hepatocytes.

The combination of increased mitochondrial membrane potential and increased NADH relates to an enhancement of bioenergetic capacity of the mitochondria when silica hydride is present in the cell suspension. Silica hydride makes electrons or H— available to cofactors able to utilize these for cellular energy production. NADH provides electrons to the mitochondrial electron transport chain, directly producing H₂O and ATP, the primary cellular energy source for numerous biochemical reactions throughout the cell.

It will be understood by those skilled in the art that a wide range of effective doses of silica hydride, ascertainable without undo experimentation, enhance mitochondrial bioenergetic capacity in humans to a greater or lesser degree. Such range includes doses as small as about 10 mg per day, or doses as large as about 5000 mg per day. The doses may be ingested any number of times per day. Additionally, individuals may find it necessary to increase or decrease the amount of silica hydride ingested to achieve desired results. For example, an individual may find 75 mg of silica hydride per day more beneficial than 10 mg. Similarly, one may find that increasing the dosage to 150 mg per day, 250 mg per day, or even 400 mg per day, and so on, achieves progressively better results. On the other hand, where one individual sees peak results taking 5000 mg per day at various intervals, another may see similar results by ingesting 3500 mg per day at a different set of intervals. Likewise, it may be advantageous to limit the dose, say from 3500 mg per day to 2000 mg per day, or to 1000 mg per day, or even to 750 mg per day, in order to see if the same results may be obtained at lower dosage levels.

Silica hydride minerals have also been tested for, and shown, antioxidant characteristics. In one evaluation, tests on silica hydride were conducted using electron spin resonance techniques (ESR) by a scientist who specializes in the evaluation of antioxidants at a major university. The following is quoted from the test report:

We have made a thorough investigation of the antioxidant activity of [silica hydride] by several experimental methods. Hydroxyl radical scavenging activity was found in two different [silica hydride] preparations provided.

Our conclusions are that [silica hydride] has antioxidant activity towards hydroxyl radicals. Hydroxyl radicals are among the most dangerous of oxygen free radicals that occur in biological systems. They are the same types of radicals that can be produced by exposure to ionizing radiation. Therefore, it can be stated that [silica hydride] has antioxidant activity in this regard.

(Lester Packer, Ph.D., University of California at Berkeley, 1999).

In a second evaluation of the antioxidant characteristics of silica hydride, tests were conducted by another scientist who specializes in the evaluation of antioxidants at a separate major university. The following is quoted from the test report:

When assayed in the standard assay for super oxide dismutase activity based upon the reduction of cytochrome c by xanthine (see J. Biol. Chem. 244: 6049-6055, 1969), silica hydride mineral showed two characteristics:

1) Silica hydride can directly reduce cytochrome c, showing that it is a reducing agent (or an antioxidant).

2) Silica hydride can inhibit the superoxide-mediated reduction of cytochrome c, indicating that it can scavenge the superoxide free radical.

(Joe McCord, Ph.D., University of Colorado Health Sciences Center, 1998).

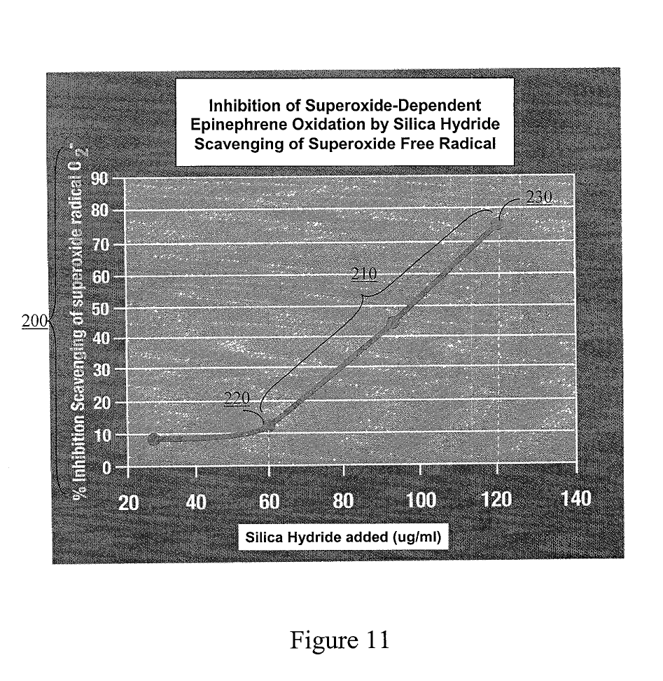

FIG. 11 shows the results of one evaluation of silica hydride's antioxidant capabilities. In this test, the silica hydride mineral was assayed with an alternative method based on the ability of superoxide to oxidize epinephrine to adrenochrome. As FIG. 11 demonstrates, silica hydride's antioxidant effects (200) increased linearly (210) from concentrations of less than 60 micrograms/ml (220) to greater than 120 micrograms/ml (230).

It will be understood by those skilled in the art that a wide range of effective doses of silica hydride, ascertainable without undo experimentation, increase antioxidant activity in humans to a greater or lesser degree. Such range includes doses as small as about 10 mg per day, or doses as large as about 5000 mg per day. The doses may be ingested any number of times per day. Additionally, individuals may find it necessary to increase or decrease the amount of silica hydride ingested to achieve desired results. For example, an individual may find 75 mg of silica hydride per day more beneficial than 10 mg. Similarly, one may find that increasing the dosage to 150 mg per day, 250 mg per day, or even 400 mg per day, and so on, achieves progressively better results. On the other hand, where one individual sees peak results taking 5000 mg per day at various intervals, another may see similar results by ingesting 3500 mg per day at a different set of intervals. Likewise, it may be advantageous to limit the dose, say from 3500 mg per day to 2000 mg per day, or to 1000 mg per day, or even to 750 mg per day, in order to see if the same results may be obtained at lower dosage levels.

Silica hydride minerals are also capable of making water more suitable for conversion into optimal extracellular and intracellular body fluids. Tap water and bottled waters are oxidized, acidic, and do not provide a source of electrons. When consumed, the body must substantially alter their chemical characteristics in order to convert water into optimal extracellular and intracellular body fluids. Three such characteristics are conductivity, oxidation-reduction potential (ORP) and surface tension.

Conductivity is critical to the optimal function of body fluids, and the conductivity of typical tap water is much lower than desirable. When conductivity was assessed, 250 mg of silica hydride in 8 oz of tap water increased conductivity from 10 to 895 μMhos/c. Total dissolved solids also increased from 30 ppm to 400-450 ppm.

Oxidation-reduction potential is a measure of negative ions in solution. Metabolic oxidation-reduction reactions are extremely important to the cell. Oxidation-reduction reactions (or “redox” reactions) involve the loss of electrons by one chemical species, becoming oxidized, and the gain of electrons by another species, becoming reduced. The flow of electrons in redox reactions is responsible, directly or indirectly, for all of the work done by living organisms. The tendency of these reactions to occur depends upon the relative affinity of the electron acceptor for electrons. Electron flow is spontaneous and exergonic (releases energy) in the cell because oxygen, the final electron acceptor, has a higher affinity for electrons than do the intermediates that donate electrons.

ORP is a measure of oxidation-reduction potential with standard meters, such as the Electronmeter, that measure negative ions in solution. Various types of water, depending on mineral salt content, and water treatments will show variability in ORP readings with standard meters. ORP measurements, when silica hydride is added to water, vary depending on water purity, dilution, the time it has remained in solution, proper cleaning of the meter probes, and maintenance of the meter.

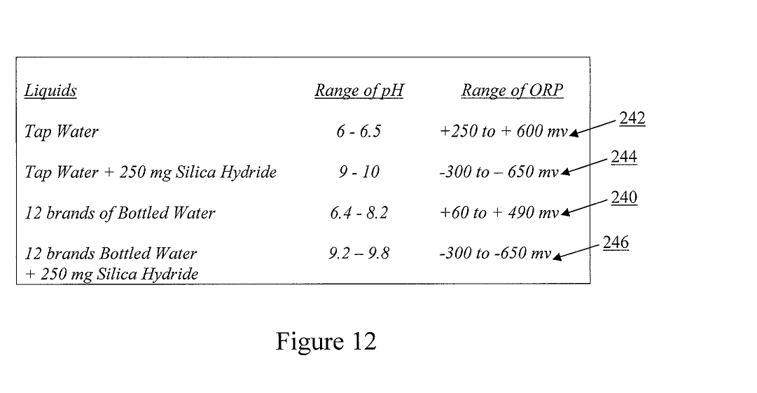

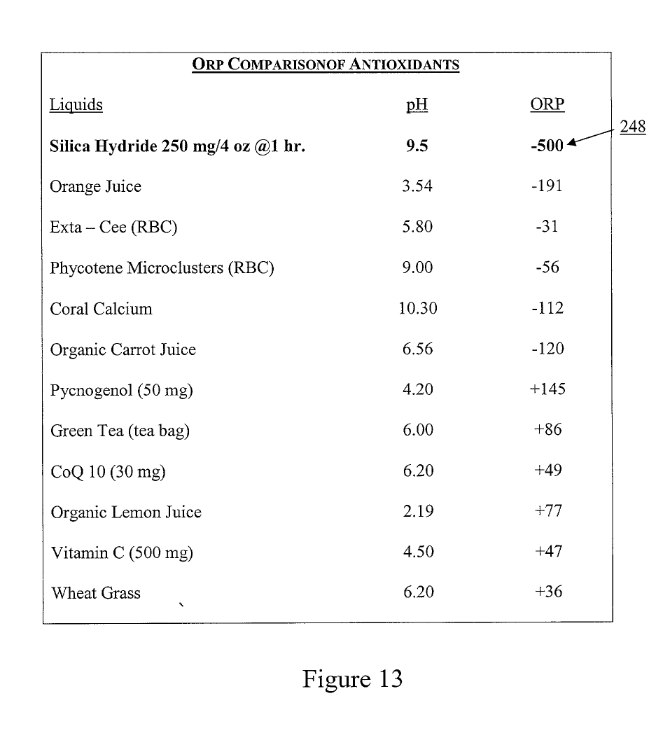

To measure the level of electrons available in silica hydride, a 250 mg capsule was dispersed into 8 oz of tap water and into each of twelve 8 oz. glasses of different brands of bottled water. The results shown in FIG. 12 were measured before, and one hour after, the addition of silica hydride. The average ORP of the bottled waters (240), +60 to +490 mv, was lower and more desirable than the ORP of tap water (242), +250 to +600 mv. In contrast, the average ORP of both tap water and bottled water after addition of 250 mg of silica hydride (244), (246), −300 to −650 mv for both, was dramatically lower. FIG. 13 compares the ORP of 250 mg of silica hydride mineral in 4 oz. of water (248) to that of other common antioxidants.

In addition to conductivity and oxidation-reduction potential, surface tension is a third property of concern when comparing the characteristics of tap or bottled water to the characteristics of optimal body fluids. The surface tension of tap water is approximately 73 dynes. The surface tension of extracellular fluids is much lower at approximately 45 dynes. This low surface tension is critical to cellular function, absorption of nutrients, and to the removal of toxins. 250 mg of silica hydride dispersed into an 8 oz. glass of tap water has been shown to reduce surface tension to approximately 45 dynes, the same surface tension as extracellular fluids.

A wide range of doses of silica hydride, readily ascertainable by those skilled in the art, will make water more suitable for conversion into optimal intra- and extra-cellular body fluids. Such range includes amounts as small as about 1 mg per 8 oz. of tap water, or amounts as large as about 5000 mg per 8 oz. of tap water. Individuals may find it necessary to increase or decrease the amount in order to achieve desired results. For example, an individual may find 30 mg of silica hydride per 8 oz. of tap water more beneficial than 1 mg. Similarly, one may find that increasing the dosage to 100 mg, 200 mg, or even 400 mg per 8 oz. of tap water, and so on, achieves progressively better results. On the other hand, where one individual sees peak results by including 5000 mg of silica hydride in 8 oz. of tap, another may see similar results by mixing 3500 mg per 8 oz. of tap water. Likewise, it may be advantageous to limit the dose, say from 3500 mg, to 2000 mg, to 1000 mg, or even to 750 mg per 8 oz. of tap water, in order to see if the same results may be obtained at lower dosage levels.

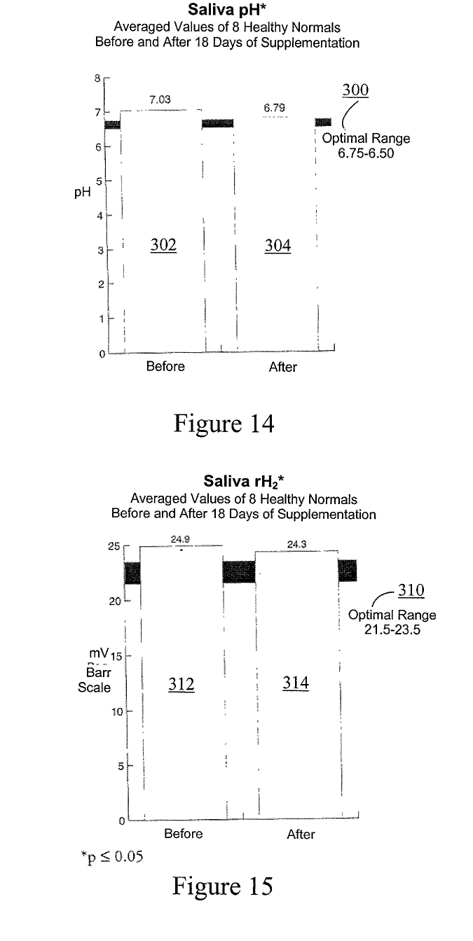

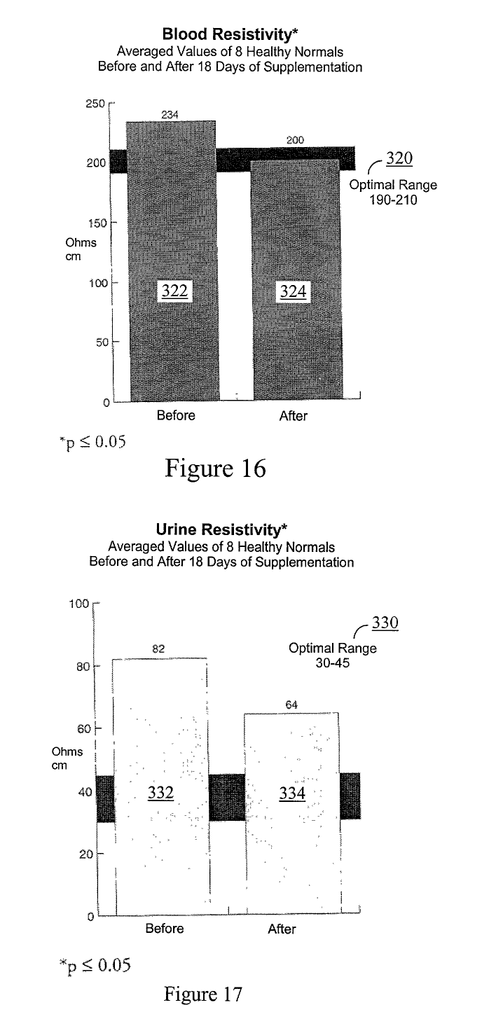

Other tests of silica hydride also showed that in eight normal adult subjects, supplementation with silica hydride improved the characteristics of body fluids. As shown in FIGS. 14 through 17, respectively, saliva pH, saliva rH₂, blood resistivity, and urine resistivity showed statistically significant improvement in eight subjects after eighteen days of supplementation with four 250 mg doses of silica hydride per day on a schedule of one in the morning, two at midday, and one in the evening. FIG. 14 shows that the optimal range for saliva pH is 6.75 to 6.50 (300) and that the supplementation decreased the group's average saliva pH from 7.03 (302) to 6.79 (304). FIG. 15 shows that the optimal range for saliva rH₂ is 21.5 to 23.5 (310) and the supplementation decreased the group's average saliva rH₂ from 24.9 (312) to 24.3 (314). The optimal range for blood resistivity, as shown by FIG. 16, is 190 to 210 (320), and the supplementation accounted for an average decrease of from 234 (322) to 200 (324). The results of the urine resistivity tests are shown in FIG. 17, which reflects an optimal range from 30 to 45 (330) and a decrease in the average value for the group after supplementation from 82 (332) to 64 (334).

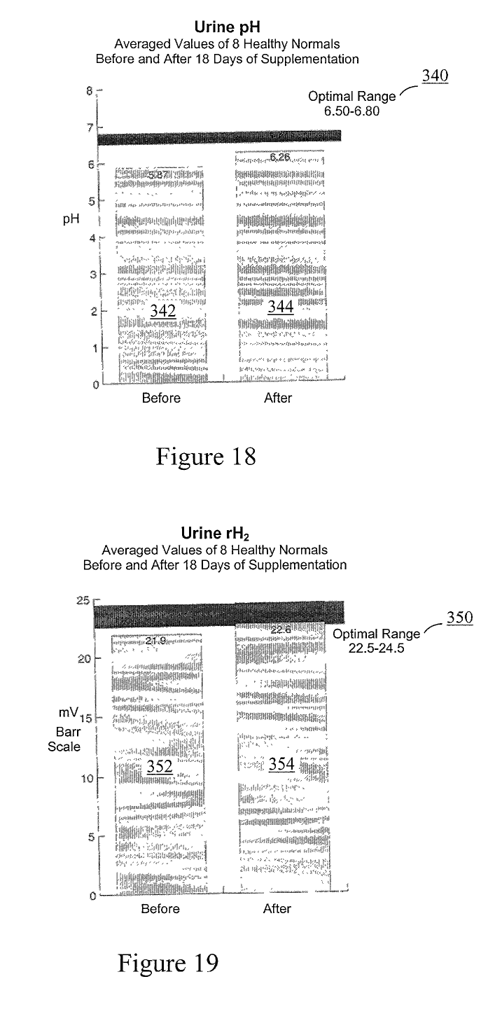

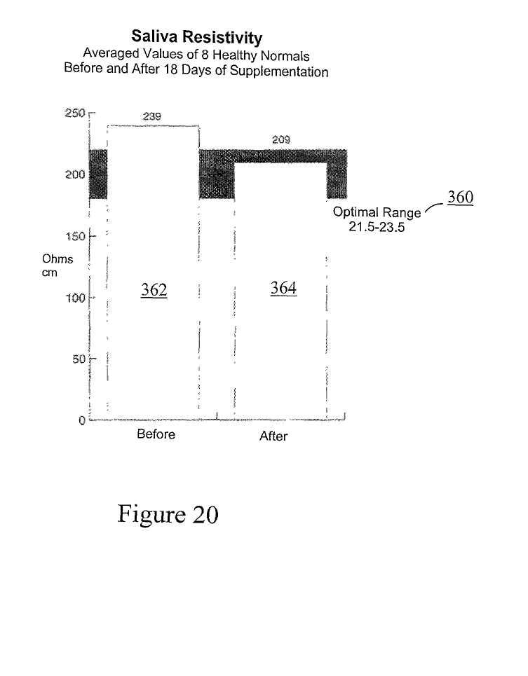

FIGS. 18 through 20 reflect data for three other parameters, urine pH, urine rH₂, and saliva resistivity, respectively, that also showed improvement over the 18-day supplementation period when their average changes were calculated. The optimal range for urine pH is reported from 6.50 to 6.80 (340). The described supplementation with silica hydride increased the average urine pH value from 5.87 (342) to 6.26 (344). The optimal range for Urine rH₂ is reported from 22.5 to 24.5 (350). The silica hydride supplementation increased average urine rH₂ values from 21.9 (352) to 22.6 (354). As for saliva resistivity, FIG. 20 shows the optimal range is reported from 21.5 to 23.5 (360) and the average values for the group decreased from 239 (362) to 209 (364) with supplementation.

The preceding effective dose of silica hydride for improving the characteristics of certain body fluids calls for 250 mg, four times per day. It will be understood by those skilled in the art, however, that a wide range of doses, ascertainable without undo experimentation, produce similar beneficial effects to a greater or lesser degree. Alternative embodiments for improving the characteristics of certain body fluids include ingesting silica hydride in amounts as small as about 10 mg per day, or amounts as large as about 5000 mg per day. In addition, the doses may be ingested any number of times per day, as infrequently as once, or several more than the above-mentioned four times per day. Individuals may also find it necessary to increase or decrease the amount of silica hydride ingested to achieve desired results. For example, an individual may find 75 mg of silica hydride per day more beneficial than 10 mg. Similarly, one may find that increasing the dosage to 150 mg per day, 250 mg per day, or even 400 mg per day, and so on, achieves progressively better results. On the other hand, where one individual sees peak results taking 5000 mg per day at various intervals, another may see similar results by ingesting 3500 mg per day at a different set of intervals. Likewise, it may be advantageous to limit the dose, say from 3500 mg per day to 2000 mg per day, or to 1000 mg per day, or even to 750 mg per day, in order to see if the same results may be obtained at lower dosage levels.

Although preferred embodiments of the present invention have been described in the foregoing Detailed Description and illustrated in the accompanying drawings, it will be understood that the invention is not limited to the embodiments disclosed, but is capable of numerous modifications without departing from the spirit of the invention. Accordingly, the present invention is intended to encompass such modifications as fall within the scope of the appended claims.

We claim:

1. A method of reducing lactic acid buildup during and after exercise comprising ingesting an effective dose of silica hydride mineral.

2. The method of claim 1 wherein said step of ingesting an effective dose of silica hydride comprises ingesting from about 10 mg to about 5000 mg of silica hydride per day.

3. The method of claim 1 wherein said step of ingesting an effective dose of silica hydride comprises ingesting from about 75 mg to about 3500 mg of silica hydride per day.

4. The method of claim 1 wherein said step of ingesting an effective dose of silica hydride comprises ingesting from about 150 mg to about 2000 mg of silica hydride per day.

5. The method of claim 1 wherein said step of ingesting an effective dose of silica hydride comprises ingesting from about 250 mg to about 1000 mg of silica hydride per day.

6. The method of claim 1 wherein said step of ingesting an effective dose of silica hydride comprises ingesting from about 400 mg to about 750 mg of silica hydride per day.

7. A method of increasing cellular hydration comprising ingesting an effective dose of silica hydride mineral.

8. The method of claim 7 wherein said step of ingesting an effective dose of silica hydride comprises ingesting from about 10 mg to about 5000 mg of silica hydride per day.

9. The method of claim 7 wherein said step of ingesting an effective dose of silica hydride comprises ingesting from about 75 mg to about 3500 mg of silica hydride per day.

10. The method of claim 7 wherein said step of ingesting an effective dose of silica hydride comprises ingesting from about 150 mg to about 2000 mg of silica hydride per day.

11. The method of claim 7 wherein said step of ingesting an effective dose of silica hydride comprises ingesting from about 250 mg to about 1000 mg of silica hydride per day.

12. The method of claim 7 wherein said step of ingesting an effective dose of silica hydride comprises ingesting from about 400 mg to about 750 mg of silica hydride per day.

13. A method of reducing free radical damage comprising ingesting an effective dose of silica hydride mineral.

14. The method of claim 13 wherein said step of ingesting an effective dose of silica hydride comprises ingesting from about 10 mg to about 5000 mg of silica hydride per day.

15. The method of claim 13 wherein said step of ingesting an effective dose of silica hydride comprises ingesting from about 75 mg to about 3500 mg of silica hydride per day.

16. The method of claim 13 wherein said step of ingesting an effective dose of silica hydride comprises ingesting from about 150 mg to about 2000 mg of silica hydride per day.

17. The method of claim 13 wherein said step of ingesting an effective dose of silica hydride comprises ingesting from about 250 mg to about 1000 mg of silica hydride per day.

18. The method of claim 13 wherein said step of ingesting an effective dose of silica hydride comprises ingesting from about 400 mg to about 750 mg of silica hydride per day.

19. A method of enhancing mitochondrial bioenergetic capacity comprising ingesting an effective dose of silica hydride mineral.

20. The method of claim 19 wherein said step of ingesting an effective dose of silica hydride comprises ingesting from about 10 mg to about 5000 mg of silica hydride per day.

21. The method of claim 19 wherein said step of ingesting an effective dose of silica hydride comprises ingesting from about 75 mg to about 3500 mg of silica hydride per day.

22. The method of claim 19 wherein said step of ingesting an effective dose of silica hydride comprises ingesting from about 150 mg to about 2000 mg of silica hydride per day.

23. The method of claim 19 wherein said step of ingesting an effective dose of silica hydride comprises ingesting from about 250 mg to about 1000 mg of silica hydride per day.

24. The method of claim 19 wherein said step of ingesting an effective dose of silica hydride comprises ingesting from about 400 mg to about 750 mg of silica hydride per day.

25. A method of increasing antioxidant activity comprising ingesting an effective dose of silica hydride mineral.

26. The method of claim 25 wherein said step of ingesting an effective dose of silica hydride comprises ingesting from about 10 mg to about 5000 mg of silica hydride per day.

27. The method of claim 25 wherein said step of ingesting an effective dose of silica hydride comprises ingesting from about 75 mg to about 3500 mg of silica hydride per day.

28. The method of claim 25 wherein said step of ingesting an effective dose of silica hydride comprises ingesting from about 150 mg to about 2000 mg of silica hydride per day.

29. The method of claim 25 wherein said step of ingesting an effective dose of silica hydride comprises ingesting from about 250 mg to about 1000 mg of silica hydride per day.

30. The method of claim 25 wherein said step of ingesting an effective dose of silica hydride comprises ingesting from about 400 mg to about 750 mg of silica hydride per day.

31. A method of making water more suitable for conversion into optimal extracellular and intracellular body fluids comprising ingesting water mixed with an effective dose of silica hydride mineral.

32. The method of claim 31 wherein said step of ingesting water mixed with an effective dose of silica hydride comprises ingesting from about 1 mg to about 5000 mg of silica hydride per 8 ounces of water.

33. The method of claim 31 wherein said step of ingesting water mixed with an effective dose of silica hydride comprises ingesting from about 30 mg to about 3500 mg of silica hydride per 8 ounces of water.

34. The method of claim 31 wherein said step of ingesting water mixed with an effective dose of silica hydride comprises ingesting from about 100 mg to about 2000 mg of silica hydride per 8 ounces of water.

35. The method of claim 31 wherein said step of ingesting water mixed with an effective dose of silica hydride comprises ingesting from about 250 mg to about 1000 mg of silica hydride per 8 ounces of water.

36. The method of claim 31 wherein said step of ingesting water mixed with an effective dose of silica hydride comprises ingesting from about 400 mg to about 750 mg of silica hydride per 8 ounces of water.

37. A method of improving the characteristics of body fluids, such as saliva pH, saliva rH₂, blood resistivity, urine resistivity, urine pH, urine rH₂, and saliva resistivity comprising ingesting an effective dose of silica hydride mineral.

38. The method of claim 37 wherein said step of ingesting an effective dose of silica hydride comprises ingesting from about 10 mg to about 5000 mg of silica hydride per day.

39. The method of claim 37 wherein said step of ingesting an effective dose of silica hydride comprises ingesting from about 75 mg to about 3500 mg of silica hydride per day.

40. The method of claim 37 wherein said step of ingesting an effective dose of silica hydride comprises ingesting from about 150 mg to about 2000 mg of silica hydride per day.

41. The method of claim 37 wherein said step of ingesting an effective dose of silica hydride comprises ingesting from about 250 mg to about 1000 mg of silica hydride per day.

42. The method of claim 37 wherein said step of ingesting an effective dose of silica hydride comprises ingesting from about 400 mg to about 750 mg of silica hydride per day.
